(19) United States
(12) Patent Application Publication
(10) Pub. No.: US 2007/0156155 A1
(43) Pub. Date: Jul. 5, 2007

(54) Surgical cup impactor

(76) Inventor: Brad A. Parker, Warsaw, IN (US)

Correspondence Address: TAYLOR & AUST, P.C., 142 SOUTH MAIN STREET, P. O. BOX 560, AVILLA, IN, 46710, US

(21) Appl. No.: 11/324,683

(22) Filed: Jan. 3, 2006

(51) Int. Cl. A61F 2/00

(52) U.S. Cl. 606/91

(57) ABSTRACT

A surgical tool including a direct drive shaft and a frame. The frame including an angled portion. The direct drive shaft is at least partially enclosed in the angled portion and extends beyond the angled portion.

### BACKGROUND OF THE INVENTION

1. Field of the Invention

The present invention relates to a surgical instrument, and, more particularly, to a surgical cup impaction device with a release mechanism.

2. Description of the Related Art

Replacement joint assemblies that replace diseased, damaged or degraded bone are known. For example, the acetabulum of a hip may be replaced with an acetabular cup assembly that includes a metal shell component for attachment to an acetabulum to replace the natural socket. A polymeric bearing component is disposed in the metal shell component to provide a hemispherical bearing surface for receiving a replacement femur implant. Proper positioning of the acetabular cup assembly usually requires the reaming of the acetabulum to define a suitable bone cavity shape and size. The sizing of the bone cavity may be of a size comparable to the cup assembly, or slightly smaller in order to hold the shell component therein. During the surgical procedure the surgeon selects an orientation desired for the cup assembly and utilizes a positioning/impactor device to hold the bearing component at the desired orientation relative to the bone cavity and the cup assembly is driven into the bone cavity.

What is needed in the art is a cup impactor with an effective release mechanism to separate the cup assembly from the impactor once the cup assembly is positioned in a bone cavity.

### SUMMARY OF THE INVENTION

The present invention provides a surgical impactor including an offset shaft that is rotatably connected to an implantable cup assembly.

The invention comprises, in one form thereof, a surgical tool including a direct drive shaft and a frame. The frame includes an angled portion with the direct drive shaft at least partially enclosed in the angled portion and extending beyond the angled portion.

An advantage of the present invention is that a cup assembly is separated from the impactor by a rotational motion.

Another advantage of the present invention is that the drive shaft extends from an angled portion of the impactor providing an ergonomically positioned knob for rotation by a surgeon.

Yet another advantage of the present invention is that the handle of the impactor and the knob of the drive shaft are angularly offset, thereby enabling the surgeon to hold the impactor in position and easily access the rotatable release mechanism for the cup assembly.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of an embodiment of a surgical cup impactor of the present invention;

FIG. 2 is a side view of the surgical cup impactor of FIG. 1; and

FIG. 3 is a sectional view of the surgical cup impactor of FIGS. 1 and 2.

Corresponding reference characters indicate corresponding parts throughout the several views. The exemplifications set out herein illustrate one preferred embodiment of the invention, in one form, and such exemplification is not to be construed as limiting the scope of the invention in any manner.

### DETAILED DESCRIPTION OF THE INVENTION

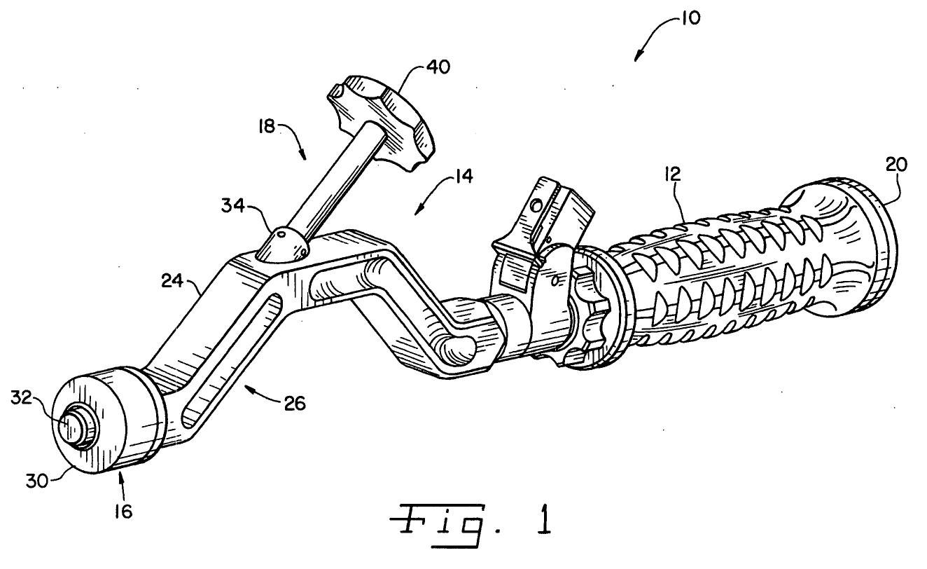

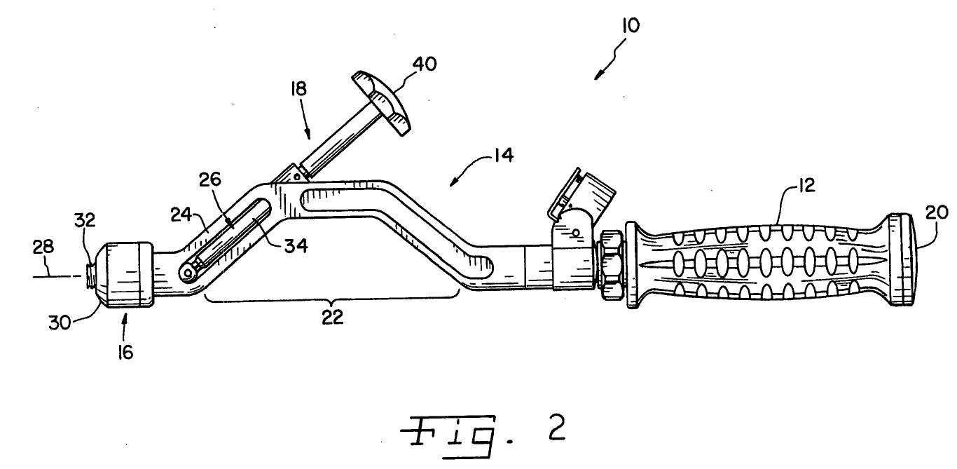

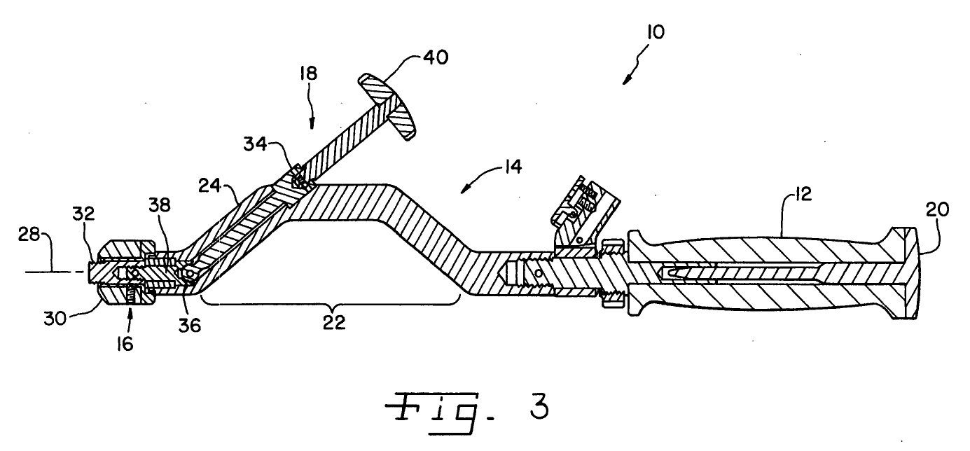

Referring now to the drawings, and more particularly to FIGS. 1-3, there is shown an embodiment of a surgical cup impactor assembly 10, including a handle 12, a frame 14, a cup interface 16 and a drive assembly 18. Handle 12 has a cushioned outer grip for easy grasping by a surgeon. At an end of handle 12 an impactor end 20 is positioned to allow an impacting device, such as a hammer, to hit the end thereof. Another end of handle 12 is connected to frame 14 so that impact force may be transferred from impactor end 20 to frame 14.

Frame 14 includes an offset 22 having at least one angled portion 24. A side opening 26 exposes a portion of drive assembly 18 when impactor assembly 10 is viewed from the side. A longitudinal axis 28 defines the direction of force that is impacted on impactor end 20 and also defines the axis about which a cup assembly, not shown, connects to cup interface 16. Two ends of frame 14 are aligned along longitudinal axis 28. Offset 22 departs from longitudinal axis 28 and returns thereto along the length of frame 14. Longitudinal axis 28 runs substantially through the center of handle 12.

A cup interface 16 includes an impactor sleeve 30 and a threaded adaptor 32. Impactor sleeve 30 is shaped to allow a portion of a cup assembly, not shown, to approach or contact the surface of impactor sleeve 30. Threaded adaptor 32 is threadedly engaged with the cup assembly to position the cup assembly relative to impactor assembly 10 so as to facilitate the implantation of the cup assembly in a bone cavity reamed by a surgeon. Threaded adaptor 32 is drivingly coupled with drive assembly 18. Drive assembly 18 rotates threaded adaptor 32 so as to engage a threaded portion of the cup assembly and to disengage the cup assembly when the cup assembly is implanted in the bone cavity. The rotation of threaded adaptor 32 causes a separation between the cup assembly and impactor sleeve 30 once the cup assembly is implanted.

Drive assembly 18 includes a shaft 34, a universal joint 36, a transfer shaft 38 and a knob 40. Shaft 34 lies at an acute angle to longitudinal axis 28 and is rotationally constrained within frame 14 so as to allow the rotational movement of threaded adaptor 32 by the rotation of shaft 34, universal joint 36 and transfer shaft 38. Universal joint 36, also known as an articulated joint 36 transfers the rotational motion of shaft 34, which is not aligned with longitudinal axis 28 to a directional rotation of transfer shaft 38 about longitudinal axis 28. Knob 40 is affixed to one end of shaft 34 to allow the easy rotation of shaft 34 by the surgeon. Shaft 34 is partially contained within angled portion 24 and extends therefrom at an angle that is substantially parallel with angled portion 24.

Operation of impactor assembly 10 includes attaching a cup assembly to threaded adaptor 32 by the rotation of drive assembly 18. The cup assembly is drawn up to and against impactor sleeve 30. The cup assembly and an end of impactor assembly 10 is inserted into a surgical incision of the patient as directed to the bone cavity, previously reamed to a desired shape and size. The cup assembly is positioned at the opening of the bone cavity and the surgeon hits impactor end 20 with a driving tool, not shown. The driving force is substantially along longitudinal axis 28 causing the cup assembly to seat in the bone cavity. Once the cup assembly is seated the surgeon rotates knob 40 in a predefined direction, thereby causing threaded adaptor 32 to disengage from the cup assembly. Impactor assembly 10 is then removed from the incision.

Advantageously, the present invention includes a direct drive threaded portion that allows for easy coupling to a cup assembly and for easy release thereof once implanted. The relative angles of shaft 34 and handle 12 provide a desirable ergonomic location of the two for easy operation by the surgeon.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A surgical tool, comprising:
a direct drive shaft; and
a frame including an angled portion, said direct drive shaft at least partially enclosed in said angled portion and extending beyond said angled portion.

2. The surgical tool of claim 1, wherein said direct drive shaft includes an articulated joint.

3. The surgical tool of claim 2, further comprising a transfer shaft connected to said articulated joint.

4. The surgical tool of claim 1, further comprising a knob, said direct drive shaft having an end to which said knob is connected.

5. The surgical tool of claim 1, wherein said frame has an axis, said frame including an offset portion that departs from said axis and returns to said axis.

6. The surgical tool of claim 5, wherein said offset portion includes said angled portion as one part of said offset.

7. The surgical tool of claim 6, wherein said frame includes at least one open portion substantially exposing said direct drive shaft.

8. The surgical tool of claim 5, wherein said direct drive shaft is at an acute angle to said axis.

9. The surgical tool of claim 8, further comprising a transfer shaft rotationally aligned with said axis, said transfer shaft coupled with said direct drive shaft, said transfer shaft proximate an end of said frame.

10. The surgical tool of claim 9, further comprising an impactor end connected to an other end of said frame.

11. A surgical impactor, comprising
a shaft; and
a frame having a longitudinal axis and an angled portion that departs from said longitudinal axis, said shaft being rotatably constrained in said angled portion and extending beyond said angled portion.

12. The surgical impactor of claim 11, wherein said shaft includes an articulated joint connected to an end of said shaft.

13. The surgical impactor of claim 12, further comprising a transfer shaft connected to said articulated joint, said transfer shaft substantially aligned with said longitudinal axis.

14. The surgical impactor of claim 11, further comprising a knob, said shaft having an end to which said knob is connected.

15. The surgical impactor of claim 11, wherein said frame includes an offset portion that departs from said axis and returns to said axis.

16. The surgical impactor of claim 15, wherein said offset portion includes said angled portion as one part of said offset.

17. The surgical impactor of claim 16, wherein said frame includes at least one open portion substantially exposing said shaft.

18. The surgical impactor of claim 15, wherein said shaft is at an acute angle to said longitudinal axis.

19. The surgical impactor of claim 18, further comprising a transfer shaft rotationally aligned with said longitudinal axis, said transfer shaft coupled with said shaft, said transfer shaft proximate an end of said frame.

20. The surgical impactor of claim 19, further comprising an impactor end connected to an other end of said frame.
